(19) United States
(12) Patent Application Publication
(10) Pub. No.: US 2007/0160651 A1
(43) Pub. Date: Jul. 12, 2007

(54) Nanoemulsions

(76) Inventors: Michael Mueller, Monheim (DE); Ansgar Behler, Bottrop (DE); Stefanie Seidler, Duesseldorf (DE); Caroline Goget, Duesseldorf (DE); Ulrich Issberner, Ambler, PA (US)

Correspondence Address: COGNIS CORPORATION;PATENT DEPARTMENT, 300 BROOKSIDE AVENUE, AMBLER, PA, 19002, US

(21) Appl. No.: 10/589,772

(22) PCT Filed: Feb. 9, 2005
(86) PCT No.: PCT/EP05/01278

§ 371 Date: Aug. 17, 2006

(30) Foreign Application Priority Data
Feb. 18, 2004 (DE) 10 2004 008 107.7

(51) Int. Cl. A61K 9/70; A61K 8/73

(52) U.S. Cl. 424/443; 424/70.13; 977/926

(57) ABSTRACT

A nanoemulsion with a mean particle diameter of 20 to 250 nm including 5 to 50% by weight of at least one alkyl and/or alkenyl oligoglycoside carboxylic acid salt corresponding to formula (I): R¹O[G]ₚO[(CH₂)ₘCOO⁻X⁺]ₙ wherein R¹ is an alkyl and/or alkenyl group containing 4-22 carbon atoms, G is a sugar unit containing 5 or 6 carbon atoms, p is a number of 1-10, m is a number of 1-5, n is a number of 1-5 and X represents alkali metal, alkaline earth metal, ammonium, alkanolammonium, alkyl ammonium or glucammonium; 10 to 50% by weight of an oil component; and 0 to 15% by weight of mono- and/or polyhydric alcohols containing 1 to 4 carbon atoms, and an article or formulation containing the nanoemulsion are provided.

### RELATED APPLICATIONS

This application is a national phase filing under 35 U.S.C. § 371 of International Application No. PCT/EP2005/001278 which has an international filing date of Feb. 9, 2005, and which designated the United States of America and which claims priority to German Application No. 102004008107.7, filed Feb. 18, 2004, the entire disclosures of which are hereby incorporated herein by reference.

### FIELD OF THE INVENTION

The invention relates generally to nanoemulsions, and more particularly, to nanoemulsions including at least one alkyl and/or alkenyl oligoglycoside carboxylic acid salt, an oil component, and mono and/or polyhydric alcohols, and to an article or formulation containing the nanoemulsion.

### BACKGROUND INFORMATION

Cosmetic cleaning preparations are having to meet ever increasing consumer demands. Thus, not only are such preparations expected to show excellent foam behavior and good cleaning performance, they are also expected to care for and condition the skin and hair. The care components or skin conditioners are mostly oily substances. Oil-containing water-based surfactant formulations usually show poor foam behavior, i.e. they show poor foam generation and develop a low maximum foam volume. Because of this, ethoxylated compounds, such as Sodium Laureth Sulfate for example, are very often used as the surfactant component. However, because these compounds are increasingly falling into disrepute, there is a considerable demand for formulations that are free from ethoxylated compounds. In addition, transparent surfactant-containing preparations are enjoying great popularity, although it has proved difficult to maintain this transparency when oil components are added.

Thus, WO 98/40044 describes water-based preparations of water-soluble surfactants which contain lipid/surfactant mixed micelles with a mean particle size below 500 nm and which are white/bluish in appearance. WO 98/15255 relates to microemulsion gels of the oil-in-water type where the oil droplets are stabilized in the water phase by associative thickeners.

Accordingly, the problem addressed by the present invention was to provide water-based transparent or opaque surfactant-containing formulations with a content of oil components which would generate a high foam volume and which would show good foam behavior and good cleaning performance. In addition, the formulations would be free from ethoxylated compounds.

### SUMMARY OF THE INVENTION

Briefly described, according to an aspect of the invention, a nanoemulsion with a mean particle diameter of 20 to 250 nm includes: (a) 5 to 50% by weight of at least one alkyl and/or alkenyl oligoglycoside carboxylic acid salt corresponding to formula (I): R¹O[G]ₚO[(CH₂)ₘCOO⁻X⁺]ₙ wherein R¹ is an alkyl and/or alkenyl group containing 4 to 22 carbon atoms, G is a sugar unit containing 5 or 6 carbon atoms, p is a number of 1 to 10, m is a number of 1 to 5, n is a number of 1 to 5 and X represents alkali metal, alkaline earth metal, ammonium, alkanolammonium, alkyl ammonium or glucammonium; (b) 10 to 50% by weight of an oil component; and, (c) 0 to 15% by weight of mono- and/or polyhydric alcohols containing 1 to 4 carbon atoms, wherein the sum of components (a)+(b) makes up 10 to 55% by weight of the composition as a whole.

According to another aspect of the invention, an article may be impregnated with the nanoemulsion described above.

According to another aspect of the invention, a formulation may contain the nanoemulsion described above.

### DETAILED DESCRIPTION OF THE INVENTION

The present invention relates to nanoemulsions with a mean particle diameter of 20 to 250 nm containing
(a) 5 to 50% by weight of at least one alkyl and/or alkenyl oligoglycoside carboxylic acid salt corresponding to formula (I):
   R¹O[G]ₚO[(CH₂)ₘCOO⁻X⁺]ₙ (I)
   in which R¹ is an alkyl and/or alkenyl group containing 4 to 22 carbon atoms, G is a sugar unit containing 5 or 6 carbon atoms, p is a number of 1 to 10, m is a number of 1 to 5, n is a number of 1 to 5 and X stands for alkali metal, alkaline earth metal, ammonium, alkanolammonium, alkyl ammonium or glucammonium,
(b) 10 to 50% by weight of an oil component and
(c) 0 to 15% by weight of mono- and/or polyhydric alcohols containing 1 to 4 carbon atoms,
   the sum of components (a)+(b) making up 10 to 55% by weight of the composition as a whole.

At least one alkyl and/or alkenyl oligoglycoside carboxylic acid salt corresponding to formula (I):
R¹O[G]ₚO[(CH₂)ₘCOO⁻X⁺]ₙ (I)
in which R¹ is an alkyl and/or alkenyl group containing 4 to 22 carbon atoms, G is a sugar unit containing 5 or 6 carbon atoms, p is a number of 1 to 10, m is a number of 1 to 5, n is a number of 1 to 5 and X stands for alkali metal, alkaline earth metal, ammonium, alkanolammonium, alkyl ammonium or glucammonium, is used as surfactant component (a). R¹ is preferably an alkyl and/or alkenyl group containing 12 to 18 and, more particularly, 12 to 14 and/or 16 to 18 carbon atoms and n is a number of 1 to 3.

They may be obtained by the relevant methods of preparative organic chemistry, for example by reaction of alkyl and/or alkenyl oligoglycosides with halocarboxylic acids in an alkaline medium in the presence of solvents. The alkyl and/or alkenyl oligoglycoside carboxylic acid salts may be derived from aldoses or ketoses containing 5 or 6 carbon atoms, preferably from glucose. Accordingly, the preferred alkyl and/or alkenyl oligoglycoside carboxylic acid salts are salts of alkyl and/or alkenyl oligoglucoside carboxylic acids. The index p in general formula (I) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Salts of alkyl and/or alkenyl oligoglycoside carboxylic acids with a mean degree of oligomerization of 1.1 to 3.0 are preferably used. Alkyl and/or alkenyl oligoglycoside carboxylic acid salts with a degree of oligomerization of less than 1.7 and, more particularly, between 1.2 and 1.4 are preferred from the applicational perspective.

The alkyl or alkenyl group R¹ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. More particularly, the alkyl or alkenyl group R¹ is derived from primary alcohols containing 12 to 22, preferably 12 to 18 and more particularly 12 to 14 and 16 to 18 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucoside ether carboxylic acid salts based on hydrogenated C_{12/14} coconut oil fatty alcohol having a DP of 1 to 3 are preferred.

In addition, the alkyl and/or alkenyl oligoglycoside carboxylic acid salts are preferably derived from carboxylic acids, salts or esters thereof, where m is a number of 1 to 5, preferably 2 to 4 and more particularly 1 to 2, n is a number of 1 to 5 and preferably 1 to 3 and X stands, for example, for potassium, ammonium, triethanolammonium and preferably sodium. Suitable carboxylic acids, salts and esters thereof are any of the compounds known to the expert, preferably acetic acid, salts thereof, more particularly sodium or potassium salts, or esters thereof, preferably containing 1 to 4 carbon atoms. In a preferred embodiment of the invention, the alkyl and/or alkenyl oligoglycoside carboxylic acid salts may be obtained by reaction of an aqueous solution of alkyl and/or alkenyl oligoglycosides (for example 20 to 70% by weight solutions, based on the active substance content) under nitrogen with w-halocarboxylic acid, a salt or ester thereof, preferably potassium or sodium chloroacetate (MCA), in the presence of alkali, for example alkali metal hydroxides or alkali metal carbonates, at temperatures of 50 to 100° C. The alkyl and/or alkenyl oligoglycoside is preferably reacted with the ω-halocarboxylic acid, salt or ester, preferably potassium or sodium monochloroacetate (MCA), in a molar ratio of 1:0.5 to 1:5 and preferably in a molar ratio of 1:1 to 1:3. In addition, a molar ratio of alkali to w-halocarboxylic acid, salt or ester of 1:0.5 to 1:1.5 and preferably 1:1.1 is preferably selected. The reaction of C_{12/14} alkyl and/or alkenyl oligoglycosides is preferably carried out in the absence of organic solvents. C_{16/18} alkyl and/or alkenyl oligoglycoside carboxylic acid salts are preferably prepared in the presence of C_{16/18} fatty alcohols, more particularly 1,2-propylene glycol. Alkyl and/or alkenyl oligoglycoside carboxylic acid salts such as these are distinguished by high foam strength and by their mildness towards the skin and hair.

In another particularly preferred embodiment, surfactant component (a) is a mixture of at least one alkyl and/or alkenyl oligoglycoside carboxylic acid salt corresponding to formula (I):
R¹O[G]ₚO[(CH₂)ₘCOO⁻X⁺]ₙ (I)
in which R¹ is an alkyl and/or alkenyl group containing 4 to 22, preferably 12 to 18 and more particularly 12 to 14 and/or 16 to 18 carbon atoms, G is a sugar unit containing 5 or 6 carbon atoms, p is a number of 1 to 10, m is a number of 1 to 5, n is a number of 1 to 5 and X stands for alkali metal, alkaline earth metal, ammonium, alkanolammonium, alkyl ammonium or glucammonium, and a surfactant selected from the group consisting of anionic, cationic, nonionic, zwitterionic and amphoteric surfactants.
Anionic Surfactants

Typical examples of anionic surfactants are soaps, alkyl benzene-sulfonates, alkanesulfonates, olefin sulfonates, α-methyl ester sulfonates, sulfofatty acids, alkyl sulfates, alkyl ether sulfates, mono- and dialkyl sulfosuccinates, mono- and dialkyl sulfosuccinamates, sulfotriglycerides, monoglyceride sulfates, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products). Acyl glutamates and salts thereof and alkyl and/or alkenyl sulfates are particularly preferred.

Cationic Surfactants

Typical examples of cationic surfactants are quaternary ammonium compounds and esterquats, more particularly quaternized fatty acid trialkanolamine ester salts.

Nonionic Surfactants

Typical examples of nonionic surfactants are alk(en)yl oligoglycosides, fatty acid-N-alkyl glucamides, polyol fatty acid esters, sugar esters, sorbitan esters, polysorbates, alcohol ethoxylates and amine oxides. Preferred nonionic surfactants are alkyl and/or alkenyl oligoglucosides corresponding in particular to formula (II):
R²O-[G]ₚ (I)
where R² is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry. The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (II) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view.

The alkyl or alkenyl group R² may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl group R² may also be derived from primary alcohols containing 12 to 22 and preferably 12 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} coconut oil fatty alcohol having a DP of 1 to 3 are preferred.

Alcohol ethoxylates are known from their production as fatty alcohol or oxoalcohol ethoxylates and preferably correspond to formula (III):
R¹O(CH₂CH₂O)ₙH (III)
in which R¹ is a linear or branched alkyl and/or alkenyl group containing 6 to 22 carbon atoms and n is a number of 1 to 50. Typical examples are adducts of on average 1 to 50, preferably 5 to 40 and more particularly 10 to 25 mol ethylene oxide with caproic alcohol, caprylic alcohol, 2-ethylhexyl alcohol, capric alcohol, lauryl alcohol, isotridecyl alcohol, myristyl alcohol, cetyl alcohol, palmitolelyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Adducts of 10 to 40 mol ethylene oxide with technical C₁₂₋₁₈ fatty alcohols, such as for example coconut oil, palm oil, palm kernel oil or tallow fatty alcohol, are preferred.
Zwitterionic and Amphoteric Surfactants

Examples of suitable amphoteric or zwitterionic surfactants are alkyl betaines, alkyl amidobetaines, aminopropionates, aminoglycinates, imidazolinium betaines and sulfobetaines. Examples of suitable alkyl betaines are the carboxyalkylation products of secondary and, in particular, tertiary amines corresponding to formula (IV): 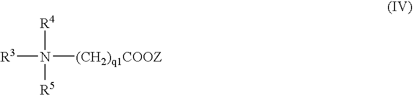
in which R³ represents alkyl and/or alkenyl groups containing 6 to 22 carbon atoms, R⁴ represents hydrogen or alkyl groups containing 1 to 4 carbon atoms, R⁵ represents alkyl groups containing 1 to 4 carbon atoms, q1 is a number of 1 to 6 and Z is an alkali metal and/or alkaline earth metal or ammonium. Typical examples are the carboxymethylation products of hexylmethyl amine, hexyldimethyl amine, octyldimethyl amine, decyl-dimethyl amine, dodecylmethyl amine, dodecyldimethyl amine, dodecyl-ethylmethyl amine, C_{12/14} cocoalkyldimethyl amine, myristyldimethyl amine, cetyldimethyl amine, stearyldimethyl amine, stearylethylmethyl amine, oleyldimethyl amine, C_{16/18} tallow alkyldimethyl amine and technical mixtures thereof.

Also suitable are carboxyalkylation products of amidoamines corresponding to formula (V): 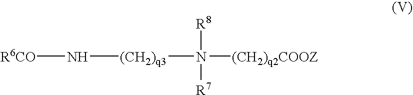
in which R⁶CO is an aliphatic acyl group containing 6 to 22 carbon atoms and 0 or 1 to 3 double bonds, R⁷ is hydrogen or represents alkyl groups containing 1 to 4 carbon atoms, R⁸ represents alkyl groups containing 1 to 4 carbon atoms, q2 is a number of 1 to 6, q3 is a number of 1 to 3 and Z is again an alkali metal and/or alkaline earth metal or ammonium. Typical examples are reaction products of fatty acids containing 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, with N,N-dimethylaminoethyl amine, N,N-dimethylamino-propyl amine, N,N-diethylaminoethyl amine and N,N-diethylaminopropyl amine which are condensed with sodium chloroacetate. A condensation product of C_{8/18}-cocofatty acid-N,N-dimethylaminopropyl amide with sodium chloroacetate is preferably used.

Imidazolinium betaines may also be used. These compounds are also known compounds which may be obtained, for example, by cyclizing condensation of 1 or 2 mol fatty acid with polyfunctional amines such as, for example, aminoethyl ethanolamine (AEEA) or diethylenetriamine. The corresponding carboxyalkylation products are mixtures of different open-chain betaines. Typical examples are condensation products of the fatty acids mentioned above with AEEA, preferably imidazolines based on lauric acid or—again —C_{12/14} cocofatty acid which are subsequently betainized with sodium chloroacetate.

The surfactant component (a) is preferably used in a quantity of 10 to 35% by weight, based on the formulation as a whole.

The oil component (b) is selected both from nonpolar and polar oils or mixtures thereof. These include, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicones, silicon methicone types, etc.) and/or aliphatic or naphthenic hydrocarbons, for example squalane, squalene or dialkyl cyclohexanes, or silicone oils or Hydrogenated Polydecene which is particularly preferred.

However, the oil component (b) may also be selected from solid fats and/or waxes which may also be present in the form of mixtures with the oils mentioned in the preceding paragraph. Typical examples of fats are glycerides, i.e. solid or liquid, vegetable or animal products which consist essentially of mixed glycerol esters of higher fatty acids. Solid mono- and diglycerides, for example glycerol monooleate or glycerol monostearate, are particularly mentioned in this regard. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable. Tocopherols and essential oils are also suitable as for use as the oil component (b).

The oil component (b) is preferably used in a quantity of 15 to 35% by weight, based on the composition as a whole. The size of the dispersed particles is in the range from 20 to 250 nm. In order to obtain the preparations according to the invention, the sum of surfactant component and oil component in the formulations should be in the range from 0.5 to 55% by weight and is preferably in the range from 2 to 40% by weight.

The nanoemulsion may optionally contain mono- or polyhydric C₁₋₄ alcohols in a quantity of 0 to 15% by weight, based on the formulation as a whole. Alcohols from the group consisting of ethanol, glycerol, ethylene glycol and/or propylene glycol are preferred. The addition of these alcohols increases the uptake capacity of the nanoemulsion for oils. In addition, the refractive index of the water phase can be assimilated to that of the dispersed oil phase, so that possible clouding is reduced. Also, the storage stability of the emulsion at low temperatures, for example at −5° C., is increased.

The emulsions can be thickened by polymers. The compounds known as associative thickeners, such as PEG-120 Methyl Glucose Dioleate or PEG-150-Distearate, are particularly preferred in this regard.

The nanoemulsions are distinguished in particular by their simple production. They are produced by stirring all the components at room temperature or, preferably where solid components are used, by pre-melting and homogenizing the components solid at room temperature and then stirring with the surfactant-containing water phase in the presence of heat. The homogenizing process is generally accelerated by heating. Temperatures in the range from 50 to 70° C. have proved to be suitable. A simple stirrer is sufficient for homogenizing. Since the oil components (b) can be present as solid substances at room temperature, the resulting emulsions can be solids dispersions in these cases.

The nanoemulsions according to the invention can be cold-stirred with other formulations and, in this way, effect the incorporation of oil components in cosmetic formulations which cannot be achieved by conventional methods. Accordingly, the present invention also relates to the use of the nanoemulsions according to the invention as a compound for the production of cosmetic preparations which preferably contain oils. The preparations obtained are stable and show excellent foam behavior. With a percentage oil component content of 15 to 35%, the nanoemulsions according to the invention preferably have a turbidity value of 100 to 8,000 NTU and are therefore opaque.

The present invention also relates to wet wipes which are characterized in that they are impregnated with a nanoemulsion according to the invention. These wet wipes are sheets based on paper, nonwovens or wovens which are coated with a nanoemulsion according to the invention and are used for body care and personal hygiene. To enable the nanoemulsions to be readily applied on an industrial scale, it is of advantage to use nanoemulsions with a water content of at least 60% by weight, preferably 70% by weight and more particularly more than 75% by weight, based on the composition of the emulsion as a whole, for coating.

The sheets thus treated can be aftertreated in a drying step to reduce the water content after application or to obtain substantially water-free products (dry wipes).

### EXAMPLES

TABLE 1
nanoemulsions

| | Examples | | | | | |
|---|---|---|---|---|---|---|
| | 1 | 2 | 3 | 4 | 5 | 6 |
| Lauryl Glucose Carboxylate (and) | 6.30 | 12.00 | 12.00 | 10.10 | 9.50 | 9.58 |
| Lauryl Glucoside (Plantapon ® | | | | | | |
| LGC SORB) | | | | | | |
| Cocamidopropyl Betaine | 3.78 | — | — | — | — | — |
| Disodium Cocoyl Glutamate | — | — | — | 0.98 | 0.51 | 0.60 |
| Glyceryl Oleate | 6.11 | 8.00 | 8.00 | 6.08 | 6.11 | 6.13 |
| Dicaprylyl Ether | 17.86 | — | — | | 17.88 | 4.47 |
| Octyldodecanol | 5.95 | — | — | 4.90 | 5.97 | 5.95 |
| Diethylhexyl cyclohexane | — | 20 | 15 | 17.25 | — | — |
| Hydrogenated Polydecene | — | — | — | — | — | 13.40 |
| Paraffin | — | — | 5 | — | — | — |
| Water | q.s. | q.s. | q.s. | q.s. | q.s. | q.s. |
| Viscosity [mPas]* | 950 | 720 | 800 | 850 | 830 | 120 |
| Particle size** | 123 (b) | 140 | 148 | 132 | 108 (b) | 136 (b) |

*Cone/plate viscosimeter (C-VOR Bohlin Instruments), T = 25° C., shear rate 10 s⁻¹
**Mean particle diameter in nm; (a) as measured with a Horiba LB-500 particle size analyzer (principle: dynamic light scattering); (b) as measured with a Coulter LS 230 particle size analyzer (principle: laser diffraction)

### Example 7

### Use on Sheet-Form Materials

Water and preservatives were added at room temperature to the nanoemulsions obtained in Examples 1 to 6. The wetting solutions formed are particularly suitable as sprayable lotions for cleansing wipes, preferably for the face and for babies' skin. 3 g of the wetting solution per g wipe were applied by impregnation or spraying.

Composition of the Wetting Solutions:

| | |
|---|---|
| Nanoemulsions of Examples 1 to 6 | 20% by weight |
| Water | 79% by weight |
| Euxyl ® K702 | 1% by weight |
| Citric acid | q.s. (pH = 5.5) |

### Example 8

### Use as a Compound for Surfactant-Containing Preparations

The nanoemulsions obtained in Examples 2, 3 and 5 were stirred at room temperature into a surfactant-containing pre-formulation. No special stirrer was needed. If the surfactant-containing formulations are transparent, the formulations obtained by incorporation of the nanoemulsions appear transparent to slightly cloudy, depending on the quantity of nanoemulsion incorporated. In addition, the preparations obtained show high stability in long-term tests. Additional experiments, where attempts were made to incorporate the pure oil or wax components in the surfactant-containing pre-formulation, all produced negative results. The incorporation of the nanoemulsions according to the invention in surfactant-containing formulations is thus an elegant method of stably incorporating oils and waxes that cannot be directly incorporated in surfactant-containing preparations. The composition of the surfactant-containing pre-formulation of Sodium Laureth Sulfate and Cocoamidopropyl Betaine was selected so that the Sodium Laureth Sulfate and Cocamidopropyl Betaine contents in the final formulation were 9% by weight and 3% by weight, respectively. If, for example, the nanoemulsions 2, 3 and 5 from Table 1 are stirred into the surfactant-containing pre-formulation, the following preparations are obtained:

TABLE 3
cosmetic preparations

| | A | B | C | D |
|---|---|---|---|---|
| Nanoemulsion from Tab. 1 used | 2 | 3 | 5 | 5 |
| Sodium Laureth Sulfate | 9.00 | 9.00 | 9.00 | 9.00 |
| Cocamidopropyl Betaine | 3.00 | 3.00 | 3.00 | 3.00 |
| Lauryl Glucose Carboxylate (and) Lauryl | 1.29 | 1.29 | 0.63 | 1.27 |
| Glucoside (Plantapon ® LGC SORB) | | | | |
| Disodium Cocoyl Glutamate | — | — | 0.03 | 0.07 |
| Glyceryl Oleate | 0.86 | 0.86 | 0.41 | 0.82 |
| Dicaprylyl Ether | — | — | 1.19 | 2.39 |
| Octyldodecanol | — | — | 0.40 | 0.80 |
| Diethylhexyl cyclohexane | 2.14 | 1.61 | — | — |
| Paraffin | — | 0.54 | — | — |
| Polyquaternium 10 | 0.20 | 0.20 | 0.20 | 0.20 |
| PEG-150 Distearate* | 1.00 | 1.00 | 1.00 | 1.00 |
| Euxyl ® K400 | 0.10 | 0.10 | 0.10 | 0.10 |

| | |
|---|---|
| Water | to 100 |

*optional

The Examples shown in Table 3 may optionally be adjusted to a pH favorable to cosmetic applications with appropriate acids/bases. The Formulation Examples shown in Table 3 are eminently suitable as rinse-off products for personal care, for example as shampoos, body washes, etc.

What is claimed is:

1. A nanoemulsion with a mean particle diameter of 20 to 250 nm comprising:
(a) 5 to 50% by weight of at least one alkyl and/or alkenyl oligoglycoside carboxylic acid salt corresponding to formula (I):
R¹O[G]ₚO[(CH₂)ₘCOO⁻X⁺]ₙ (I)
wherein R¹ is an alkyl and/or alkenyl group containing 4 to 22 carbon atoms, G is a sugar unit containing 5 or 6 carbon atoms, p is a number of 1 to 10, m is a number of 1 to 5, n is a number of 1 to 5 and X represents alkali metal, alkaline earth metal, ammonium, alkanolammonium, alkyl ammonium or glucammonium;
(b) 10 to 50% by weight of an oil component; and,
(c) 0 to 15% by weight of mono- and/or polyhydric alcohols containing 1 to 4 carbon atoms,
wherein the sum of components (a)+(b) makes up 10 to 55% by weight of the composition as a whole.

2. The nanoemulsion according to claim 1, wherein the emulsion additionally contains a surfactant selected from the group consisting of anionic, cationic, nonionic, amphoteric and zwitterionic surfactants and mixtures thereof.

3. The nanoemulsion according to claim 1, wherein component (c) is selected from the group consisting of ethanol, glycerol, ethylene glycol, propylene glycol, and mixtures thereof.

4. An article impregnated with the nanoemulsion of claim 1.

5. The article according to claim 4, wherein the article is a wet wipe.

6. A cosmetic formulation incorporating the nanoemulsion according to claim 1.
